(19) United States
(12) Patent Application Publication
(10) Pub. No.: US 2024/0078625 A1
(43) Pub. Date: Mar. 7, 2024

(54) AI SERVER PROVIDING WORKER SAFETY CONTROL SOLUTION AND OPERATION METHOD OF AI SYSTEM INCLUDING THE SAME

(71) Applicant: Electronics and Telecommunications Research Institute, Daejeon (KR)

(72) Inventors: Aram LEE, Daejeon (KR); Hyun Seo KANG, Daejeon (KR); Jeong Eun KIM, Daejeon (KR); Kyeeun KIM, Daejeon (KR)

(73) Assignee: Electronics and Telecommunications Research Institute, Daejeon (KR)

(21) Appl. No.: 18/081,397

(22) Filed: Dec. 14, 2022

(30) Foreign Application Priority Data
Sep. 5, 2022 (KR) 10-2022-0111965

(51) Int. Cl. G06Q 50/26; G05B 13/02

(52) U.S. Cl. CPC G06Q 50/265; G05B 13/027

(57) ABSTRACT

Disclosed is a method of operating an artificial intelligence system that provides a worker safety control solution and includes a plurality of smart belts and an artificial intelligence server, which includes detecting, by one of the plurality of smart belts, whether a worker is buried, generating, by the smart belt, biometric data and work environment data of the worker, generating, by the smart belt, an alarm signal based on the biometric data and the work environment data, transmitting, by the smart belt, the alarm signal, the biometric data, and the work environment data to the artificial intelligence server, and generating a plurality of rescue scenarios by inferring the biometric data and the work environment data when the artificial intelligence server receives a plurality of alarm signals from the plurality of smart belts.

### CROSS-REFERENCE TO RELATED APPLICATIONS

This application claims priority under 35 U.S.C. § 119 to Korean Patent Application No. 10-2022-0111965, filed on Sep. 5, 2022 in the Korean Intellectual Property Office, the disclosures of which are incorporated by reference herein in their entireties.

### BACKGROUND

Embodiments of the present disclosure described herein relate to an artificial intelligence server and an operation method of an artificial intelligence system including the same, and more particularly, relate to an artificial intelligence server for inferring biometric data and work environment data based on a learned deep learning model, and an operation method of an artificial intelligence system including the same.

While the construction industry continues to expand with the development of the global economy, fatal accidents at construction sites are continuously increasing. The representative cause of this tendency lies in responding to situations with poor objectivity and immediacy regarding abnormalities that occur within standardized workplaces. Although most construction sites collect sensing data such as oil vapor, temperature, pressure, and gas, it is difficult to make an immediate response because a complex judgment by the person in charge of safety management is required.

Currently, work environment monitoring is being carried out through CCTV for risk management at construction sites. However, it is difficult to immediately analyze the number of CCTV and surveillance images that increase according to the scale of construction with a small number of management personnel. In addition, it is necessary to actively apply deep learning to overall sensing data such as temperature, acceleration, pressure, heartbeat, air quality as well as image analysis with respect to the environment inside buildings where time-series information is not secured from the outside.

### SUMMARY

Embodiments of the present disclosure provide an artificial intelligence server that constructs a smart belt capable of securing biometric data and work environment data and infers biometric data and work environment data based on a learned deep learning model, and an operation method of an artificial intelligence system including the same.

According to an embodiment of the present disclosure, a method of operating an artificial intelligence system that provides a worker safety control solution and includes a plurality of smart belts and an artificial intelligence server, which includes detecting, by one of the plurality of smart belts, whether a worker is buried, generating, by the smart belt, biometric data and work environment data of the worker, generating, by the smart belt, an alarm signal based on the biometric data and the work environment data, transmitting, by the smart belt, the alarm signal, the biometric data, and the work environment data to the artificial intelligence server, and generating a plurality of rescue scenarios by inferring the biometric data and the work environment data when the artificial intelligence server receives a plurality of alarm signals from the plurality of smart belts. According to an embodiment, the artificial intelligence server may include a processor including a deep learning model for inferring the biometric data and the work environment data, and the generating of the plurality of rescue scenarios may include inferring first work environment data and first biometric data, when the processor receives a first alarm signal from among the plurality of alarm signals.

According to an embodiment, the work environment data may include information on temperature, humidity, noise, and gas of a work environment, the processor may sequentially generate the plurality of rescue scenarios based on the work environment data, and the generating of the plurality of rescue scenarios may further include generating a target rescue scenario by inferring information on the gas, when the processor receives information on the gas among the work environment data.

According to an embodiment, the artificial intelligence system may further include a plurality of BLE beacons, and the transmitting of the alarm signal to the artificial intelligence server may include selecting, by the smart belt, first BLE beacons capable of transmitting a Bluetooth signal from among the plurality of BLE beacons based on work environment data of other workers, and transmitting the alarm signal together with the Bluetooth signal, to the artificial intelligence server through the first BLE beacons.

According to an embodiment, the transmitting of the biometric data and the work environment data to the artificial intelligence server may include selecting, by the smart belt, second BLE beacons different from the first BLE beacons capable of transmitting the Bluetooth signal from among the plurality of BLE beacons based on work environment data of the other workers, and transmitting the biometric data and the work environment data together with the Bluetooth signal to the artificial intelligence server through the second BLE beacons after the alarm signal is transmitted.

According to an embodiment, the generating of the target rescue scenario by inferring the information on the gas may include recognizing a gas leakage amount based on the information on the gas in the work environment, predicting the gas leakage amount in the work environment, and performing a first calculation on a difference between the recognition result and the prediction result.

According to an embodiment, the biometric data may include information on abdominal pressure, breathing rate, movement, and fall of the worker, the generating of the target rescue scenario may further include inferring information on the movement of the worker, and the inferring of the information on the movement may include recognizing a position of the worker based on the information on the movement, predicting the position of the worker, and performing a second calculation on a difference between the recognition result and the prediction result.

According to an embodiment, the target rescue scenario may be a rescue scenario first generated by the processor among the plurality of rescue scenarios based on the first calculation result and the second calculation result.

According to an embodiment of the present disclosure, an artificial intelligence server which provides a worker safety control solution, which includes a data collection platform that collects work environment data and biometric data of a worker from a plurality of smart belts, and a processor that generates a plurality of rescue scenarios based on the biometric data and the work environment data when alarm signals are received from the plurality of smart belts, and the alarm signals are generated based on the work environment data and the biometric data, the work environment data includes information on temperature, humidity, noise, and gas of a work environment, and the biometric data includes information on abdominal pressure, breathing rate, movement, and fall of the worker.

According to an embodiment, when a first alarm signal among the alarm signals is received, the processor may infer first biometric data and first work environment data included in the first alarm signal.

According to an embodiment, the processor may sequentially generate the plurality of rescue scenarios based on the biometric data and the work environment data, and when information on the gas among the work environment data is received, the processor may generate a target rescue scenario by inferring the information on the gas.

According to an embodiment, the processor may receive one of a rescue signal or a distress signal from each of the plurality of smart belts before generating the plurality of rescue scenarios after receiving the alarm signals, and the one signal may be determined by each of the plurality of smart belts based on the biometric data of the worker.

According to an embodiment, the inferring of the information on the gas may include recognizing a gas leakage amount based on the information on the gas in the work environment, predicting the gas leakage amount in the work environment, and performing a first calculation on a difference between the recognition result and the prediction result.

According to an embodiment, the generating of the target rescue scenario may further include inferring information on the movement of the worker, and the inferring of the information on the movement may include recognizing a position of the worker based on the information on the movement, predicting the position of the worker, and performing a second calculation on a difference between the recognition result and the prediction result.

According to an embodiment, the target rescue scenario may be a rescue scenario first generated by the processor among the plurality of rescue scenarios based on the first calculation result and the second calculation result.

### BRIEF DESCRIPTION OF THE FIGURES

The above and other objects and features of the present disclosure will become apparent by describing in detail embodiments thereof with reference to the accompanying drawings.

FIG. 1 is a diagram illustrating an artificial intelligence system, according to an embodiment of the present disclosure.

FIG. 2 is a diagram illustrating a communication network of a smart belt in detail.

FIG. 3 is a diagram illustrating in detail a multi-modal sensor of a smart belt included in an artificial intelligence system.

FIG. 4A is a diagram related to an embodiment in which an alarm signal is transmitted from a smart belt to an artificial intelligence server.

FIG. 4B is a diagram related to an embodiment in which data is transmitted from a smart belt to an artificial intelligence server.

FIG. 5 is a diagram illustrating a result of inferring data through a deep learning model.

FIG. 6 is a flowchart of an operation method of a smart belt.

FIG. 7 is a flowchart of a method of operating an artificial intelligence system, according to an embodiment of the present disclosure.

FIG. 8 is a flowchart of an operation method in which an artificial intelligence server generates a target rescue scenario.

### DETAILED DESCRIPTION

Hereinafter, embodiments of the present disclosure will be described in detail and clearly to such an extent that an ordinary one in the art easily implements the present disclosure.

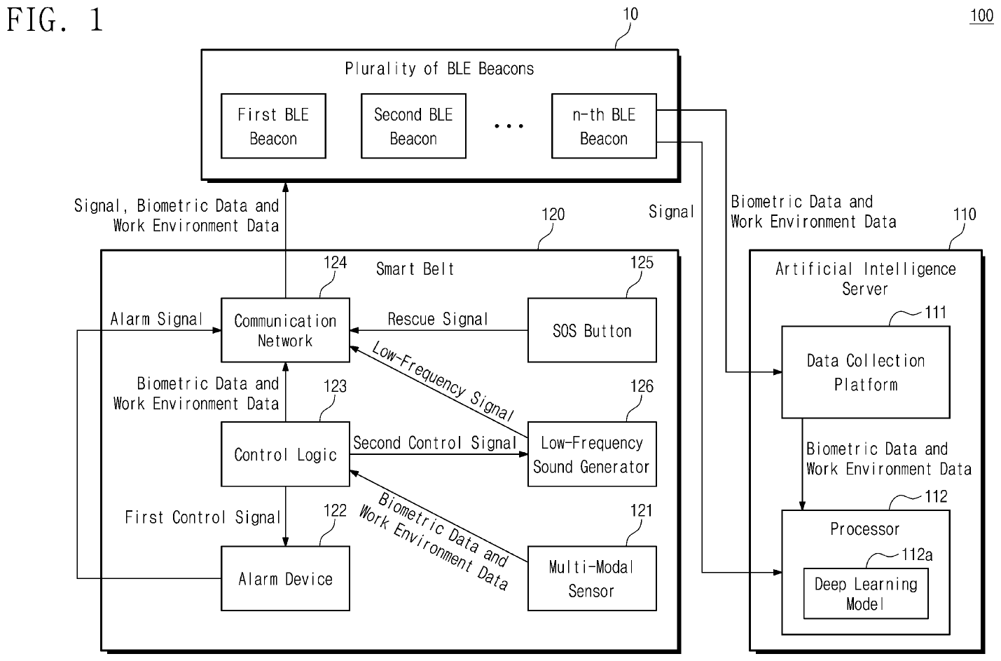

FIG. 1 is a diagram illustrating an artificial intelligence system 100, according to an embodiment of the present disclosure. Referring to FIG. 1, the artificial intelligence system 100 includes an artificial intelligence server 110 and a smart belt 120.

The artificial intelligence server 110 may include a data collection platform 111 and a processor 112.

The data collection platform 111 may collect data from external devices or external systems. The data collection platform 111 may include a memory (not illustrated) for temporarily storing received data. However, without being limited thereto, the data collection platform 111 may include any means including a storage space capable of collecting and storing data.

The processor 112 may drive operating system and applications of the artificial intelligence system 100. The processor 112 may drive the operating system and applications of the artificial intelligence system 100 by loading data from the data collection platform 111 and performing a data processing operation.

The data collection platform 111 may collect biometric data and work environment data of workers. However, without being limited thereto, the data collection platform 111 may collect all data that can be generated at the work site, such as video, image, and sensing information.

The processor 112 may generate a plurality of rescue scenarios by receiving biometric data and work environment data from the data collection platform 111. The processor 112 may include a deep learning model 112a. The deep learning model 112a may sequentially output a plurality of rescue scenarios according to types of input biometric data and work environment data. The deep learning model 112a may output a target rescue scenario when receiving specific biometric data and work environment data.

The biometric data may include information about the worker's abdominal pressure, respiratory rate, movement, and fall. The work environment data may include information on temperature, humidity, noise, and gas in a work environment in which a worker works.

However, it is not limited thereto, and the biometric data may include all biometric information that may be generated by various sensors detecting a worker's biosignal, electrical signal, and the like. As in the above description, the work environment data may include all work environment information that may be generated by various sensors detecting an inclination, pressure, and the like of the work environment.

The smart belt 120 may include a multi-modal sensor 121, an alarm device 122, a control logic 123, a communication network 124, an SOS button 125, and a low-frequency sound generator 126.

The multi-modal sensor 121 may generate biometric data of a worker and work environment data of a construction site. The multi-modal sensor 121 may obtain biometric data in real time according to changes in biometric information of a worker. The multi-modal sensor 121 may obtain work environment data in real time according to changes in environmental information of the work site.

The alarm device 122 may generate an alarm signal based on biometric data and work environment data acquired by the multi-modal sensor 121. For example, the alarm signal may be generated by comprehensively considering changes in biometric information such as a worker's respiration rate and movement, and changes in temperature, humidity, and gas distribution of a work environment. However, it is not limited thereto, and the alarm signal may be generated in consideration of changes in all biometric information and changes in all work environment information that may be detected by the aforementioned multi-modal sensor 121.

The control logic 123 may control all components of the smart belt 120 and overall operations of the smart belt 120. For example, the control logic 123 may transmit a first control signal to the alarm device 122 such that the alarm device 122 may generate an alarm signal based on the biometric data and the work environment data.

The communication network 124 may provide wired or wireless communication between the smart belt 120 and an external device. The communication network 124 may provide remote communication between smart belt 120 and an external device. The communication network 124 may use a low-power/low-speed communication protocol based on a WiFi and Ghz band RF frequencies of a Cellular. A detailed description of the configuration of the communication network 124 will be described later.

The communication network 124 may transmit an alarm signal, a worker's biometric data, and work environment data to the artificial intelligence server 110 through a plurality of BLE beacons 10 such that the processor 112 may generate rescue scenarios.

The plurality of BLE beacons 10 may be devices for BLE communication installed for each concrete section in the framework formation stage of a building. All or part of the plurality of BLE beacons 10 may be activated based on a first work environment data of the worker and a second work environment data of other workers different from the first work environment data. A detailed description of a configuration in which signals and data are transmitted through the plurality of BLE beacons 10 will be described later.

The SOS button 125 may generate a rescue signal based on a worker's input. For example, the rescue signal may be a warning sound generated by pressing the SOS button 125 when the worker is conscious. The rescue signal may or may not include the work's biometric data and work environment data.

The low-frequency sound generator 126 may generate a low-frequency signal based on the worker's biometric data. For example, the low-frequency signal may be a signal generated based on a degree of change in a worker's respiratory rate and movement when the worker loses consciousness.

The control logic 123 may transmit a second control signal to the low-frequency sound generator 126 such that the low-frequency sound generator 126 generates a low-frequency signal based on the worker's biometric data.

The control logic 123 may transmit a second control signal to the low-frequency sound generator 126 such that the low-frequency sound generator 126 does not generate a low-frequency signal when a rescue signal is generated by a worker.

One of a low-frequency signal and a rescue signal may be transmitted to an external device via the plurality of BLE beacons 10 through the communication network 124. Accordingly, the control logic 123 may control the operation of the low-frequency sound generator 126 to reduce power consumption.

When the processor 112 receives one (hereinafter referred to as signals) of an alarm signal, a rescue signal, and a low-frequency signal from the smart belt 120 through the plurality of BLE beacons 10, the processor 112 may generate a plurality of rescue scenarios through the deep learning model 112a by receiving biometric data and work environment data from the data collection platform 111.

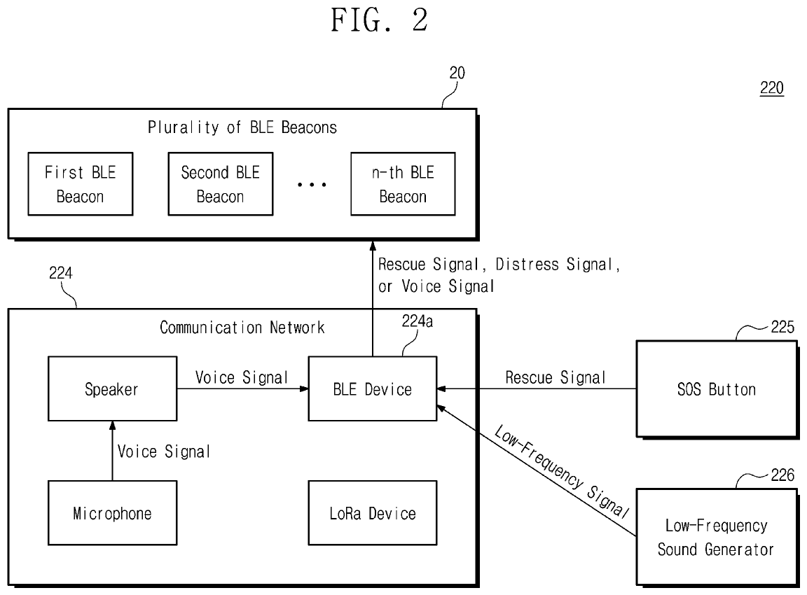

FIG. 2 is a detailed diagram of the communication network 224 of the smart belt 220. Illustratively, a communication network 224, a SOS button 225, a low-frequency sound generator 226, and a plurality of BLE beacons 20 of FIG. 2 may correspond to the communication network 124, the SOS button 125, the a low-frequency sound generator 126, and the plurality of BLE beacons 10 of FIG. 1, respectively. Therefore, additional descriptions of similar components and similar operations related thereto will be omitted to avoid redundancy.

Referring to FIGS. 1 and 2, the communication network 224 may include a BLE device 224a. When the BLE device 224a receives one of a rescue signal from the SOS button 225 and a low-frequency signal from the low-frequency sound generator 226, the BLE device 224a may transmit a signal interlinked with a Bluetooth signal to the plurality of BLE beacons 20.

When the low-frequency signal is received from the low-frequency sound generator 226, the BLE device 224a may transmit a distress signal, which is a signal in which the low-frequency signal and the Bluetooth signal are interlinked with each other, to the plurality of BLE beacons 20.

The communications network 224 may further include a microphone and a speaker. The microphone and speaker may transmit a voice signal of the worker to an external device through the plurality of BLE beacons 20.

Although not illustrated, the microphone and speaker may additionally transmit a noise signal of the work site that can be recognized at the current position of the worker when a buried accident occurs to an external device through the plurality of BLE beacons 20. In this case, the BLE device 224a may transmit a voice signal and a noise signal in conjunction with a Bluetooth signal to an external device.

The communication network 224 may further include a LoRa device. The LoRa device may generate LoRa radio waves to transmit signals from the communication network 224 to the artificial intelligence server 110 for WiFi-based communication through the plurality of BLE beacons 20.

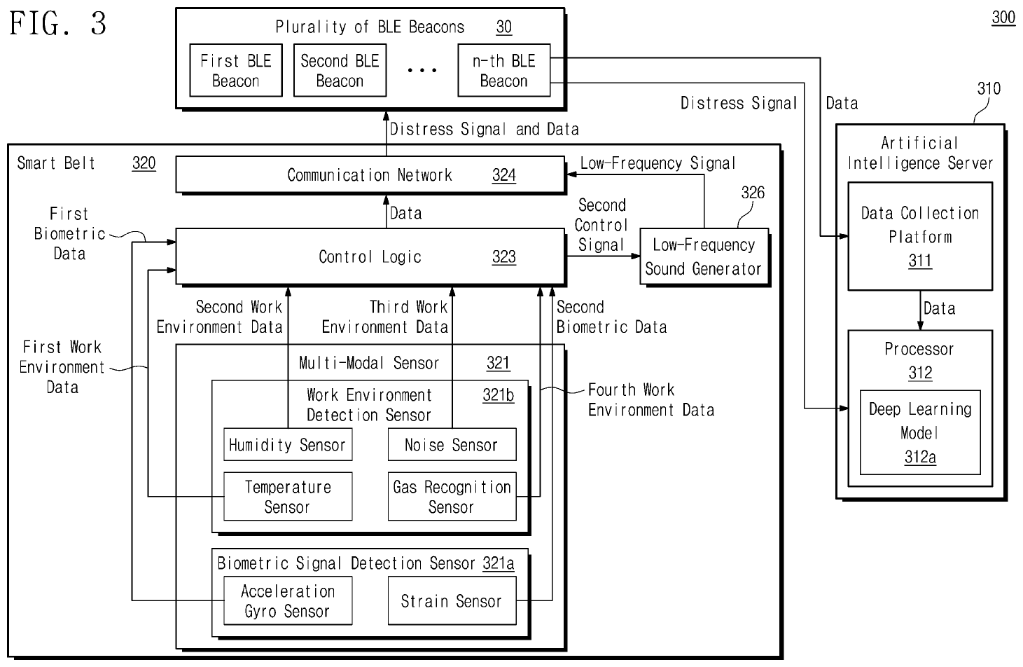

FIG. 3 is a diagram illustrating in detail a multi-modal sensor of a smart belt included in an artificial intelligence system 300. Illustratively, an artificial intelligence server 310, a smart belt 320, and a plurality of BLE beacons 30 of FIG. 3 may correspond to the artificial intelligence server 110, the smart belt 120, and the plurality of BLE beacons 10 of FIG. 1, respectively. Therefore, additional descriptions of similar components and similar operations related thereto will be omitted to avoid redundancy.

FIG. 3 illustrates the artificial intelligence system 300 when a worker loses consciousness among the embodiments mentioned in FIG. 1. Referring to FIGS. 1 and 3, a multi-modal sensor 321 may include a biometric signal detection sensor 321a and a work environment detection sensor 321b.

The biometric signal detection sensor 321a may include an acceleration gyro sensor and a strain sensor. The acceleration gyro sensor may be a sensor for acquiring information associated with movement, fall, or crash of the worker. The acceleration gyro sensor may generate first biometric data including such information.

The strain sensor may be a sensor for obtaining information associated with the abdominal pressure and respiratory rate of the worker. The strain sensor may include a plurality of fiber optic lines, and when the plurality of fiber optic lines are separately disposed above and below the smart belt 320, the strain sensor may also obtain information on a movement of the worker by analyzing a twist degree and direction of the smart belt 320. The strain sensor may generate second biometric data including such information.

The information on the movement acquired by the strain sensor may be the same as or different from the information of the movement obtained by the acceleration gyro sensor. The strain sensor may be replaced with an electronic sensor that does not include the plurality of the optic fiber lines. Although not illustrated, the biometric signal detection sensor 321a may further include other sensors capable of acquiring position information and biometric information of the worker.

The work environment detection sensor 321b may include a temperature sensor, a humidity sensor, a noise sensor, and a gas recognition sensor. The temperature sensor may generate first work environment data including information about temperature of the work environment. The humidity sensor may generate second work environment data including information about humidity of the work environment.

The noise sensor may generate third work environment data including information about noise of the work environment. The gas recognition sensor may generate fourth work environment data including information on a distribution amount of gas in the work environment. Although not illustrated, the work environment detection sensor 321b may further include other sensors capable of obtaining information on the work environment of the construction site.

A control logic 323 may transmit a second control signal to a low-frequency sound generator 326 such that the low-frequency sound generator 326 generates a low-frequency signal based on the first biometric data and the second biometric data.

The control logic 323 may transmit the first and second biometric data and the first to fourth work environment data (hereinafter referred to as data) to the plurality of BLE beacons 30 through the communication network 324. The communication network 324 may transmit a distress signal interlinked with a low-frequency signal and a Bluetooth signal to the artificial intelligence server 310 through the plurality of BLE beacons 30.

When the distress signal is received, the processor 312 may receive data collected by the data collection platform 311 and generate rescue scenarios through a deep learning model 312a. A detailed description of a configuration for inferring input data through the deep learning model 312a will be described later.

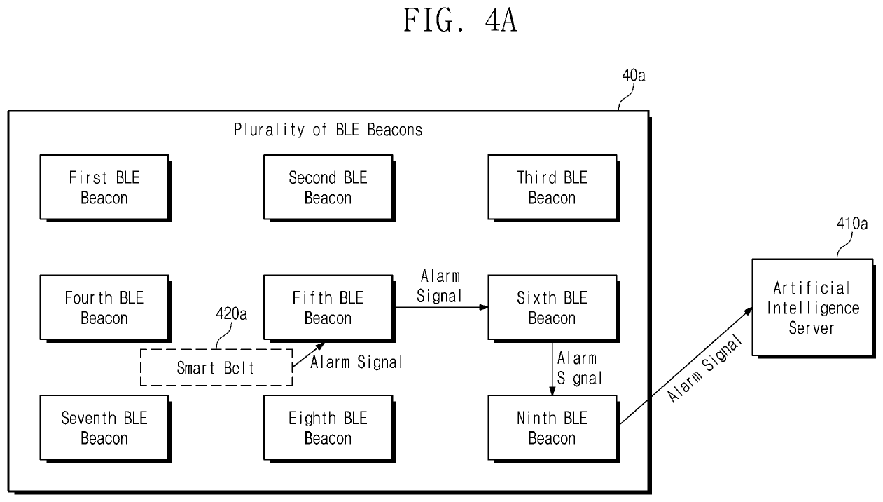

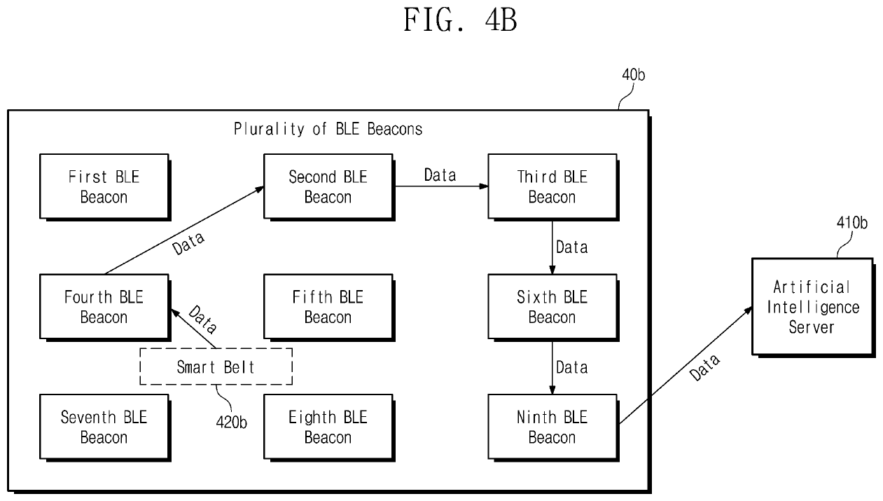

FIG. 4A is a diagram related to an embodiment in which an alarm signal is transmitted from a smart belt 420a to an artificial intelligence server 410a. Illustratively, in FIG. 4A, first to ninth BLE beacons are illustrated, but are not limited thereto, and a plurality of BLE beacons 40a may include ‘n’ (e.g., n is a natural number of 10 or more) BLE beacons. The plurality of BLE beacons 40a may form a BLE mesh network with all or some of the BLE beacons.

Referring to FIG. 4A, a worker wearing the smart belt 420a may be placed on a first BLE mesh network formed by the fourth, fifth, seventh, and eighth BLE beacons among the plurality of BLE beacons 40a when a buried accident occurs.

The fourth, fifth, seventh, and eighth BLE beacons included in the first BLE mesh network may be all or partly activated based on the worker's work environment data. For example, in FIG. 4A, only the fifth BLE beacon may be activated.

The BLE beacons not included in the first BLE mesh network may be all or partly activated based on work environment data of other workers not illustrated. For example, in FIG. 4A, sixth and ninth BLE beacons may be activated.

In this case, the alarm signal may be transmitted to the artificial intelligence server 410a via the fifth, sixth, and ninth BLE beacons.

FIG. 4B is a diagram of an embodiment in which data is transmitted from a smart belt 420b to an artificial intelligence server 410b. Illustratively, in FIG. 4B, first to ninth BLE beacons are illustrated, but are not limited thereto, and a plurality of BLE beacons 40b may include ‘n’ (e.g., n is a natural number of 10 or more) BLE beacons. Additional descriptions of components similar to those of FIG. 4A and similar operations related thereto will be omitted to avoid redundancy.

The fourth, fifth, seventh, and eighth BLE beacons included in the first BLE mesh network may be all or partly activated based on the worker's work environment data. For example, in FIG. 4B, only the fourth BLE beacon may be activated.

The BLE beacons not included in the first BLE mesh network may be all or partly activated based on work environment data of other workers not illustrated. For example, in FIG. 4B, the second, third, sixth, and ninth BLE beacons may be activated.

In this case, data may be transmitted to the artificial intelligence server 410b via the fourth, second, third, sixth, and ninth BLE beacons.

FIGS. 4A and 4B are only diagrams illustrating an example in which each of the alarm signal and data are transmitted from the smart belts 420a and 420b to the artificial intelligence servers 410a and 410b via some BLE beacons among the plurality of BLE beacons 40a and 40b. Accordingly, without being limited thereto, each of the alarm signal and data may be transmitted from the smart belts 420a and 420b to the artificial intelligence servers 410a and 410b through various paths.

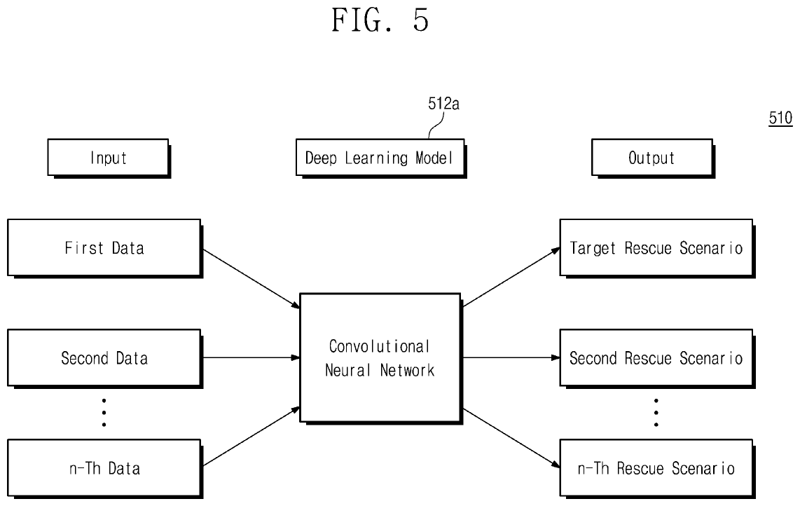

FIG. 5 is a diagram illustrating a result of inferring data through a deep learning model 512a in an artificial intelligence server 510. Referring to FIG. 5, the deep learning model 512a based on a convolutional neural network may output first to n-th rescue scenarios based on first to n-th data.

The convolutional neural network may be configured to process input data to generate output data. For reference, the convolutional neural network may be based on an R-CNN, a Fast R-CNN, a Faster R-CNN, a Mask R-CNN, or similar convolutional neural networks of various types, but is not limited to the aforementioned networks.

Each of the first to n-th data may be data obtained by combination of biometric data and work environment data of the worker. The deep learning model 512a may generate a target rescue scenario and second to n-th rescue scenarios by inferring the first to n-th data.

The deep learning model 512a may infer data included in the signals whenever signals are received. However, the deep learning model 512a may receive signals ‘n’ times, may infer data included in each signal ‘n’ times, and sequentially generate n rescue scenarios depending on biometric information and work environment information included in the data.

The ‘n’ number of rescue scenarios may include a target rescue scenario generated first among a plurality of rescue scenarios. The target rescue scenario may be a rescue scenario for a high-risk situation, such as when a worker does not move or when a gas distribution amount at a burial site near a worker exceeds a threshold value without being recognized by the worker.

For example, although not illustrated, the first data may be a combination of work environment data including information about gas and biometric data including information about a worker's movement. In this case, the deep learning model 512a may output a target rescue scenario by inferring the first data based on the learned result.

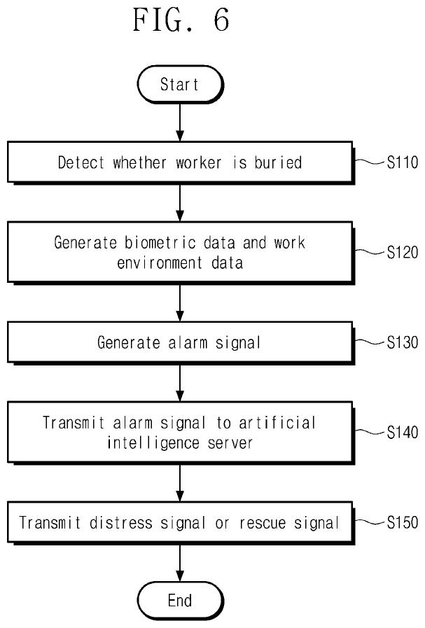

FIG. 6 is a flowchart of an operation method of a smart belt. Referring to FIGS. 1 and 6, in operation S110, the smart belt 120 may detect whether the worker is buried due to the collapse of the building based on the sensing data of the multi-modal sensor 121. The sensing data may include information about temperature, humidity, and brightness of the buried environment.

When a worker is buried by a building collapse, the worker may be positioned under the concrete rubble, and communication with the outside based on RF frequency may be impossible.

In operation S120, the multi-modal sensor 121 may obtain the worker's biometric data and work environment data. The worker's work environment data may include information about temperature, humidity, noise, and gas in the work environment, and the worker's biometric data may include information about the worker's abdominal pressure, breathing rate, movement, and fall. However, it is the same as mentioned above that the information included in each of the work environment data and biometric data is not limited thereto.

In operation S130, the alarm device 122 may generate an alarm signal in response to a control signal of the control logic 123, based on the biometric data and the work environment data. It is the same as mentioned above that the alarm signal may be generated by comprehensively considering the degree of change in biometric information and the degree of change in the work environment.

In operation S140, the communication network 124 may transmit an alarm signal to the artificial intelligence server 110. In this case, the communication network 124 may transmit an alarm signal to the artificial intelligence server 110 via activated BLE beacons among the plurality of BLE beacons 10, based on the work environment data of the worker and other workers. Accordingly, the communication network 124 may secure communication paths bypassing empty concrete spaces at the buried site.

In operation S150, the communication network 124 may transmit a low-frequency signal or a rescue signal to the artificial intelligence server 110. Although not illustrated, the communication network 124 may transmit one of a low-frequency signal or a rescue signal to the artificial intelligence server 110 via the activated BLE beacons among the plurality of BLE beacons 10, based on the work environment data of the worker and other workers. In this case, a transmission path of one of the low-frequency signal and the rescue signal may be different from the transmission path of the alarm signal.

It is the same as mentioned above that only one signal of the low-frequency signal or the rescue signal may be transmitted to the artificial intelligence server 110 under the control of the control logic 123 depending on whether the worker is conscious or not.

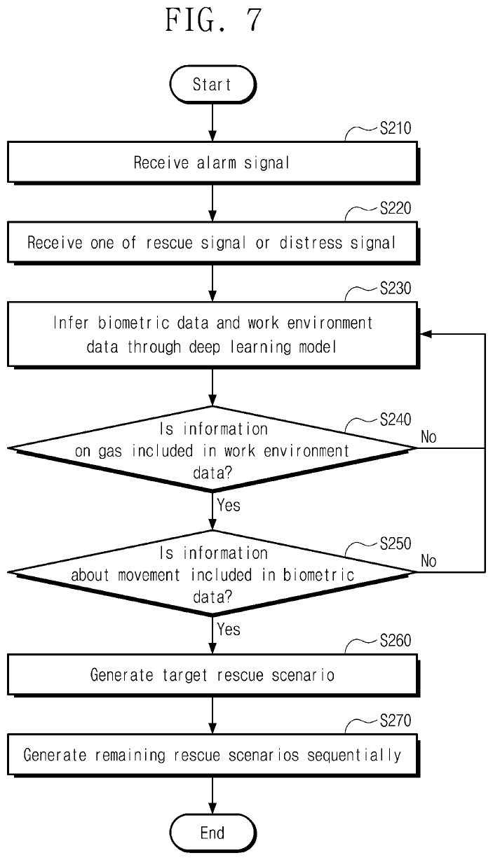

FIG. 7 is a flowchart of a method of operating an artificial intelligence system, according to an embodiment of the present disclosure. Referring to FIGS. 1 and 7, the artificial intelligence server 110 may receive an alarm signal from the smart belt 120 in operation S210.

In operation S220, the artificial intelligence server 110 may receive one of a rescue signal and the distress signal from the smart belt 120.

In operations S210 and S220, the configuration in which one signal (signals) of an alarm signal and a rescue signal, or a distress signal may be received through different paths through the plurality of BLE beacons is the same as that mentioned above.

In operation S230, the deep learning model 112a may receive biometric data and work environment data and may infer based on the convolutional neural network. The deep learning model 112a may receive biometric data and work environment data from the data collection platform 111 whenever the artificial intelligence server 110 receives the signals from the smart belt 120.

In operation S240, the deep learning model 112a may infer whether information on gas at the buried site is included in the input work environment data. When information about the gas is not included, it is inferred that the risk is low and operation S230 of inferring other biometric data and work environment data may proceed. When information about the gas is included, it is inferred that the risk is high, and operation S250 may proceed.

In operation S250, the deep learning model 112a may infer whether information about the movement of the worker is included in the received biometric data. When information about the movement is not included, it is inferred that the risk is low and operation S230 of inferring other biometric data and work environment data may proceed. When information about the movement is included, it is inferred that the risk is high, and operation S260 may proceed.

In operation S260, the deep learning model 112a may generate a target rescue scenario. The target rescue scenario may be a rescue scenario first generated among a plurality of rescue scenarios as a result of inference obtained by combining work environment data including information about gas and biometric data including information about a worker's movement.

In operation S270, the deep learning model 112a may sequentially generate second to n-th rescue scenarios. When the first to n-th data are input to the deep learning model 112a, rescue scenarios corresponding to each data may be generated. However, the number of generated rescue scenarios may be more or less than the number of pieces of data.

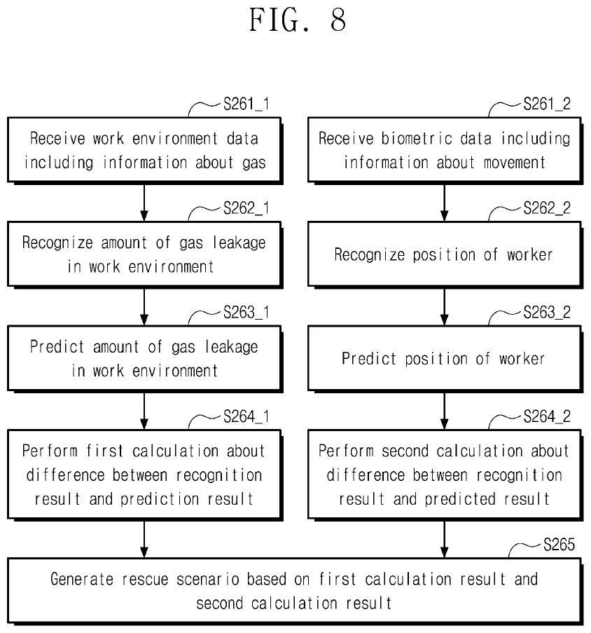

FIG. 8 is a flowchart of an operation method in which an artificial intelligence server generates a target rescue scenario. Referring to FIGS. 1 and 8, operation of generating the target rescue scenario may include operations (operations S261_1 to S264_1) of inferring information about gas based on the learned deep learning model 112a and operations (operations S261_2 to S264_2) of inferring information about the movement of the worker.

Operation of inferring information about gas may include operation of receiving work environment data including information about gas from the data collection platform 111 (S261_1), operation of recognizing, by the deep learning model 112a, the amount of gas leakage in the work environment (S262_1), operation of predicting, by the deep learning model 112a, the amount of gas leakage in the work environment (S263_1), and operation of performing, by the deep learning model 112a, a first calculation about a difference between the recognition result and the predicted result (S264_1).

In operation S264_1, operation of performing the first calculation may include obtaining a first calculation result by calculating a difference between a probability distribution of the gas leakage predicted by the deep learning model 112a and an actual probability distribution.

Operation of inferring information about the movement of the worker may include operation of receiving biometric data including information about the movement from the data collection platform 111 (S261_2), operation of recognizing the position of the worker by the deep learning model 112a (S262_2), operation of predicting the position of the worker by the deep learning model 112a (S263_2), and operation of performing, by the deep learning model 112a, a second calculation about a difference between the recognition result and the predicted result (S264_2).

In operation S264_2, operation of performing the second calculation may include obtaining a second calculation result by calculating a difference between a probability distribution of the worker's position predicted by the deep learning model 112a and an actual probability distribution.

In operation S265, the deep learning model 112a may generate a target rescue scenario based on the first calculation result and the second calculation result. Except for the rescue scenarios other than the target rescue scenario, which differ in the order of generation, the configuration inferred through the deep learning model 112a is the same as the above-mentioned configuration.

According to an embodiment of the present disclosure, an artificial intelligence server and an operation method of an artificial intelligence system including the artificial intelligence server may infer biometric data and work environment data to sequentially generate rescue scenarios. According to an embodiment of the present disclosure, it is possible to respond to timely rescue by promptly providing an appropriate rescue scenario in case of a worker's burial accident.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A method of operating an artificial intelligence system that provides a worker safety control solution and includes a plurality of smart belts and an artificial intelligence server, the method comprising:
detecting, by one of the plurality of smart belts, whether a worker is buried;
generating, by the smart belt, biometric data and work environment data of the worker;
generating, by the smart belt, an alarm signal based on the biometric data and the work environment data;
transmitting, by the smart belt, the alarm signal, the biometric data, and the work environment data to the artificial intelligence server; and
generating a plurality of rescue scenarios by inferring the biometric data and the work environment data when the artificial intelligence server receives a plurality of alarm signals from the plurality of smart belts.

2. The method of claim 1, wherein the artificial intelligence server includes a processor including a deep learning model for inferring the biometric data and the work environment data, and
wherein the generating of the plurality of rescue scenarios includes inferring first work environment data and first biometric data, when the processor receives a first alarm signal from among the plurality of alarm signals.

3. The method of claim 2, wherein the work environment data includes information on temperature, humidity, noise, and gas of a work environment,
wherein the processor sequentially generates the plurality of rescue scenarios based on the work environment data, and
wherein the generating of the plurality of rescue scenarios further includes generating a target rescue scenario by inferring information on the gas, when the processor receives information on the gas among the work environment data.

4. The method of claim 1, wherein the artificial intelligence system further includes:
a plurality of BLE beacons, and
wherein the transmitting of the alarm signal to the artificial intelligence server includes:
selecting, by the smart belt, first BLE beacons capable of transmitting a Bluetooth signal from among the plurality of BLE beacons based on work environment data of other workers; and
transmitting the alarm signal together with the Bluetooth signal, to the artificial intelligence server through the first BLE beacons.

5. The method of claim 4, wherein the transmitting of the biometric data and the work environment data to the artificial intelligence server includes:
selecting, by the smart belt, second BLE beacons different from the first BLE beacons capable of transmitting the Bluetooth signal from among the plurality of BLE beacons based on work environment data of the other workers; and
transmitting the biometric data and the work environment data together with the Bluetooth signal to the artificial intelligence server through the second BLE beacons after the alarm signal is transmitted.

6. The method of claim 3, wherein the generating of the target rescue scenario by inferring the information on the gas includes:
recognizing a gas leakage amount based on the information on the gas in the work environment;
predicting the gas leakage amount in the work environment; and
performing a first calculation on a difference between the recognition result and the prediction result.

7. The method of claim 6, wherein the biometric data includes information on abdominal pressure, breathing rate, movement, and fall of the worker,
wherein the generating of the target rescue scenario further includes inferring information on the movement of the worker, and
wherein the inferring of the information on the movement includes:
recognizing a position of the worker based on the information on the movement;
predicting the position of the worker; and
performing a second calculation on a difference between the recognition result and the prediction result.

8. The method of claim 7, wherein the target rescue scenario is a rescue scenario first generated by the processor among the plurality of rescue scenarios based on the first calculation result and the second calculation result.

9. An artificial intelligence server which provides a worker safety control solution, the artificial intelligence server comprising:
a data collection platform configured to collect work environment data and biometric data of a worker from a plurality of smart belts; and
a processor configured to generate a plurality of rescue scenarios based on the biometric data and the work environment data when alarm signals are received from the plurality of smart belts, and
wherein the alarm signals are generated based on the work environment data and the biometric data,
wherein the work environment data includes information on temperature, humidity, noise, and gas of a work environment, and
wherein the biometric data includes information on abdominal pressure, breathing rate, movement, and fall of the worker.

10. The artificial intelligence server of claim 9, wherein, when a first alarm signal among the alarm signals is received, the processor infers first biometric data and first work environment data included in the first alarm signal.

11. The artificial intelligence server of claim 9, wherein the processor sequentially generates the plurality of rescue scenarios based on the biometric data and the work environment data, and
wherein, when information on the gas among the work environment data is received, the processor generates a target rescue scenario by inferring the information on the gas.

12. The artificial intelligence server of claim 9, wherein the processor receives one of a rescue signal or a distress signal from each of the plurality of smart belts before generating the plurality of rescue scenarios after receiving the alarm signals, and
wherein the one signal is determined by each of the plurality of smart belts based on the biometric data of the worker.

13. The artificial intelligence server of claim 11, wherein the inferring of the information on the gas includes:
recognizing a gas leakage amount based on the information on the gas in the work environment;
predicting the gas leakage amount in the work environment; and
performing a first calculation on a difference between the recognition result and the prediction result.

14. The artificial intelligence server of claim 13, wherein the generating of the target rescue scenario further includes inferring information on the movement of the worker, and
wherein the inferring of the information on the movement includes:
recognizing a position of the worker based on the information on the movement;
predicting the position of the worker; and
performing a second calculation on a difference between the recognition result and the prediction result.

15. The artificial intelligence server of claim 14, wherein the target rescue scenario is a rescue scenario first generated by the processor among the plurality of rescue scenarios based on the first calculation result and the second calculation result.
